(19) United States
(12) Patent Application Publication
(10) Pub. No.: US 2007/0161855 A1
(43) Pub. Date: Jul. 12, 2007

(54) Medical procedure through natural body orifice

(75) Inventors: Takayasu Mikkaichi, Tokyo (JP); Kensei Nakahashi, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., TOKYO (JP)

(21) Appl. No.: 11/327,788

(22) Filed: Jan. 6, 2006

(51) Int. Cl. A61B 1/04; A61B 1/00

(52) U.S. Cl. 600/113; 600/175; 600/173; 600/129

(57) ABSTRACT

A medical procedure through a natural orifice according to the present invention comprises: forming an orifice in a hollow organ by using a device inserted into the hollow organ from a natural orifice of a patient; introducing a first observation device from the orifice formed in the hollow organ into an abdominal cavity; introducing a second observation device from the orifice formed in the hollow organ into the abdominal cavity; arranging the second observation device in a position different from that of the first observation device; simultaneously or selectively displaying an image obtained from the first observation device and an image obtained from the second observation device; and performing a desired procedure in the abdominal cavity while confirming the images.

### BACKGROUND OF THE INVENTION

1. Field of the Invention

The present invention relates to a medical procedure or action performed through a natural orifice of a living body.

2. Description of the Related Art

In the case where a medical procedure (including observation, treatment, and the like, which is the same hereunder) is performed for a human organ and the like, there is known a laparoscopic operation for manipulating by opening a plurality of orifices in the abdominal wall, instead of largely incising the abdominal wall, and inserting a laparoscope, a forceps, and a scalpel into the respective orifices. The laparoscopic operation can be completed simply by opening small orifices in the abdomen, having an advantage of quick recovery of the patient.

However, recently, as a method of further reducing the burden on a patient, there is proposed a procedure (or manipulation) performed by inserting a flexible endoscope from a natural orifice such as the mouth, a nostril, and the anus. An example of such a medical procedure is disclosed in U.S. Pat. No. 5,458,131. A flexible endoscope is inserted from the mouth of a patient, and the endoscope is sent out from an orifice formed in the stomach wall into the abdominal cavity. The observation of the abdominal cavity is performed by an observation device provided at the distal end of the endoscope. Furthermore, an organ is treated by using a treatment tool passed through the endoscope, and a treatment tool inserted from another orifice opened in the stomach, or inserted from the anus through an orifice opened in the lower gastrointestinal tract, into the abdominal cavity. After the procedure in the abdominal cavity is completed, the endoscope and treatment tool(s) are withdrawn, and the orifices are closed. Upon closure of an orifice, the tissue around the orifice is drawn together, and the tissue is bound up with an O-ring so as to close the orifice.

### SUMMARY OF THE INVENTION

A medical procedure through a natural orifice according to the present invention comprises: forming an orifice in a hollow organ by using a device inserted into the hollow organ from a natural orifice of a patient; introducing a first observation device from the orifice formed in the hollow organ into an abdominal cavity; introducing a second observation device from the orifice formed in the hollow organ into the abdominal cavity; arranging the second observation device in a position different from that of the first observation device; simultaneously or selectively displaying an image obtained from the first observation device and an image obtained from the second observation device; and performing a desired procedure in the abdominal cavity while confirming the images.

### BRIEF DESCRIPTION OF THE DRAWINGS

FIG. 1 is a view showing an endoscope as an example of a device used for performing a medical procedure in an embodiment, holding a capsule endoscope as a second observation device.

FIG. 2 is a perspective view of the distal end of the endoscope, showing a first observation device.

FIG. 3 is a partial cross-sectional view showing the capsule endoscope being held.

FIG. 4 is a view showing a process for holding the capsule endoscope.

FIG. 5 is a cross-sectional view showing the construction of the capsule endoscope.

FIG. 6 is an explanatory diagram of a procedure, showing a patient laid on his back.

FIG. 7 is a view showing the endoscope inserted into the stomach.

FIG. 8 is a view showing the endoscope introduced from an orifice formed in the stomach into the abdominal cavity.

FIG. 9 is a view showing the capsule endoscope that is pushed out.

FIG. 10 is a view showing the capsule endoscope attached to a magnet disposed outside of the abdominal wall.

FIG. 11 is a view showing a confirmation of a target site by the capsule endoscope and the first observation device of the endoscope.

FIG. 12 is a view showing an example of a display by having an image of the capsule endoscope and an image of the first observation device superposed.

FIG. 13 is an explanatory diagram of a procedure of treating a treatment target site by a forceps passed through a work channel.

FIG. 14 is a view showing an orifice sutured after the endoscope and the capsule endoscope are brought back into the stomach.

FIG. 15 is a view showing the endoscope attached with a small scope having a second observation device.

FIG. 16 is a view showing the small scope curved in the abdominal cavity.

FIG. 17 is a view showing the second observation device provided on the distal end of an overtube.

FIG. 18 is a view showing the second observation device provided on a rising member of an overtube.

FIG. 19 is a cross-sectional view taken along the line A-A of FIG. 18.

FIG. 20 is a view showing the rising member raised from the position of FIG. 19.

### DETAILED DESCRIPTION OF THE INVENTION

Hereunder is a detailed description of embodiments. In the following description, the same reference symbols are used for the same components, and duplicate description is omitted.

### FIRST EMBODIMENT

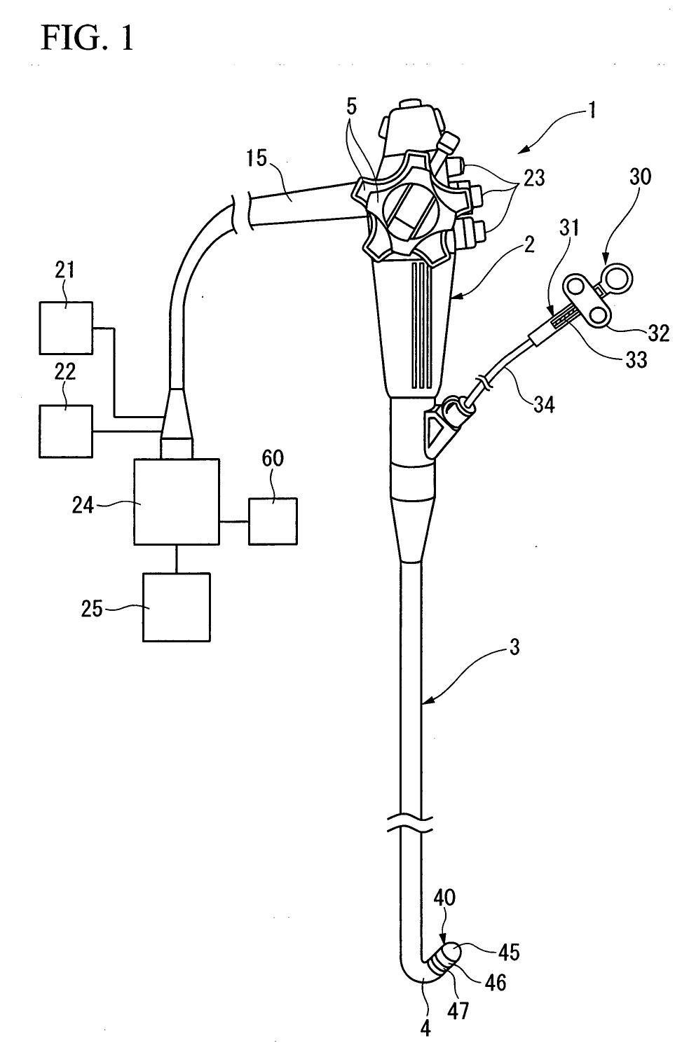

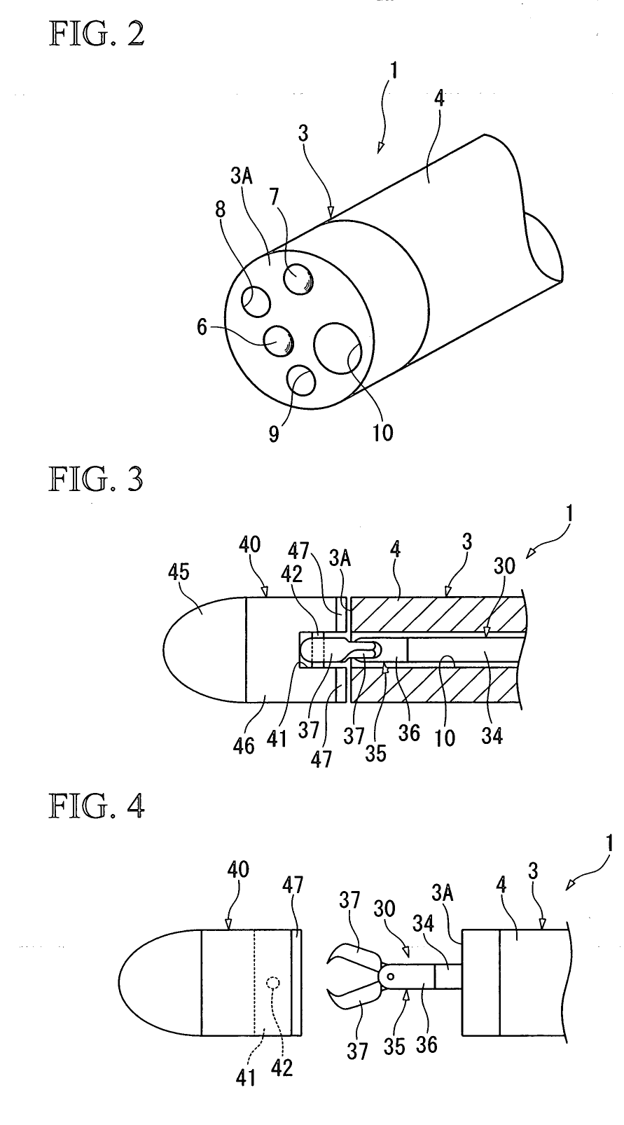

FIG. 1 shows a flexible endoscope (hereunder, called an endoscope) serving as a device used in the present embodiment, holding a capsule endoscope at the distal end of an insertion portion thereof. The endoscope 1 has an extending insertion portion 3 which is to be inserted into a patient's body from an operation portion 2 operated by an operator. The insertion portion 3 is slender and flexible. A distal end 4 of the insertion portion 3 can be curved by angle knobs 5 of the operation portion 2. As shown in FIG. 2, a distal face 3A of the insertion portion 3 is arranged with an illuminating device 7 and a first observation-device 6 for observing inside the body. The first observation device 6 comprises, for example, an observational optical system such as an object lens, and a CCD (Charged Coupled Device) as an imager. The illuminating device 7 has a construction where illuminating light is guided from a light source unit outside of the body, by means of an optical fiber. The construction may be such that an output signal from the imager is transmitted to a controller 24 described later through a signal wire passed through the insertion portion 3. Moreover, the construction may be such that an output signal from the imager is transmitted to the controller 24 by wireless means. Furthermore, the illuminating device 7 may be constructed using a publicly known light emission element (for example, light emitting diode) or the like.

Moreover, the distal face 3A of the insertion portion 3 is arranged with distal orifices of various channels 8 to 10. A fluid supply channel 8 is a duct used for supplying a fluid into the body. A suction channel 9 is a duct used for sucking a fluid from the body. A work channel 10 is a channel for work involving passing a treatment tool therethrough. The respective channels 8 to 10 are extended from the insertion portion 3 toward the operation portion 2. However, the construction of the endoscope 1 is not limited to this. For example, the construction may be such that the suction channel 9 is omitted and suction is performed by using the work channel 10. Moreover, a plurality of work channels 10 may be provided.

The proximal orifice of the work channel 10 is provided on the side of the operation portion 2. The other channels 8 and 9 are connected to a fluid supply device 21 and a suction device 22 through a universal cable 15 shown in FIG. 1. The supply of a fluid or suction thereof can be operated by buttons 23 arranged on the operation portion 2. The endoscope 1 is also connected to the controller 24, through the universal cable. The controller 24 is a device which controls the endoscope 1, and is installed with an image processor and a light source, being capable of outputting various images (images) to a monitor 25.

FIG. 1 shows a grasping forceps 30 as an example of a treatment tool passed through the work channel 10 of the endoscope 1. The grasping forceps 30 has a forceps operation portion 31 operated by an operator, and the forceps operation portion 31 is attached with a handle 32 in-a back-and-forth movable manner. The handle 32 is fixed with a wire 33. The wire 33 is led into a flexible forceps insertion portion 34 extending to the distal end of the forceps operation portion 31. As shown in FIG. 3, this forceps insertion portion 34 is passed through the work channel 10. The distal end of the forceps insertion portion 34 is provided with a treatment portion 35. As shown in FIG. 3 and FIG. 4, the treatment portion 35 has a construction where a pair of forceps members 37 is supported on a supporting portion 36 so as to be opened and closed. The pair of forceps members 37 is connected to the wire 33, and can be opened and closed according to a back-and-forth movement of the handle 32.

Here, the treatment portion 35 of the grasping forceps 30 holds a capsule endoscope 40 as a second observation device in the present embodiment. The capsule endoscope 40 has an outline of a cylindrical shape with a spherical distal end. The proximal face is provided with a radially extending groove 41 in a concave manner. The groove 41 is fixed with a pin 42 so as to transverse the groove 41. This pin 42 is held by the forceps members 37 serving as a holding part of the grasping forceps 30, by which the capsule endoscope 40 is held by the grasping forceps 30. The width and the depth of the groove 41 are in a size which allows insertion and withdrawal of the treatment portion 35. As shown in FIG. 1 and FIG. 3, the capsule endoscope 40 is abutted against the distal face 3A of the insertion portion 3. The outer diameter there of is not greater than the outer diameter of the distal end 4 of the insertion portion 3 so as not to interfere with insertion into the body.

As shown in FIG. 1 and FIG. 3, the capsule endoscope 40 has an approximately hemispherical transparent hood 45 on the distal end. Furthermore, it has a capsule type casing 46 having a flat proximal end except for the groove 41. The proximal end of the casing 46 is fixed with a pair of semicircular magnetic bodies 47 so as to avoid the groove 41. The magnetic body 47 may be a hard magnetic body material which generates a magnetic force by itself, or a soft magnetic body which is magnetized when exposed to a magnetic field.

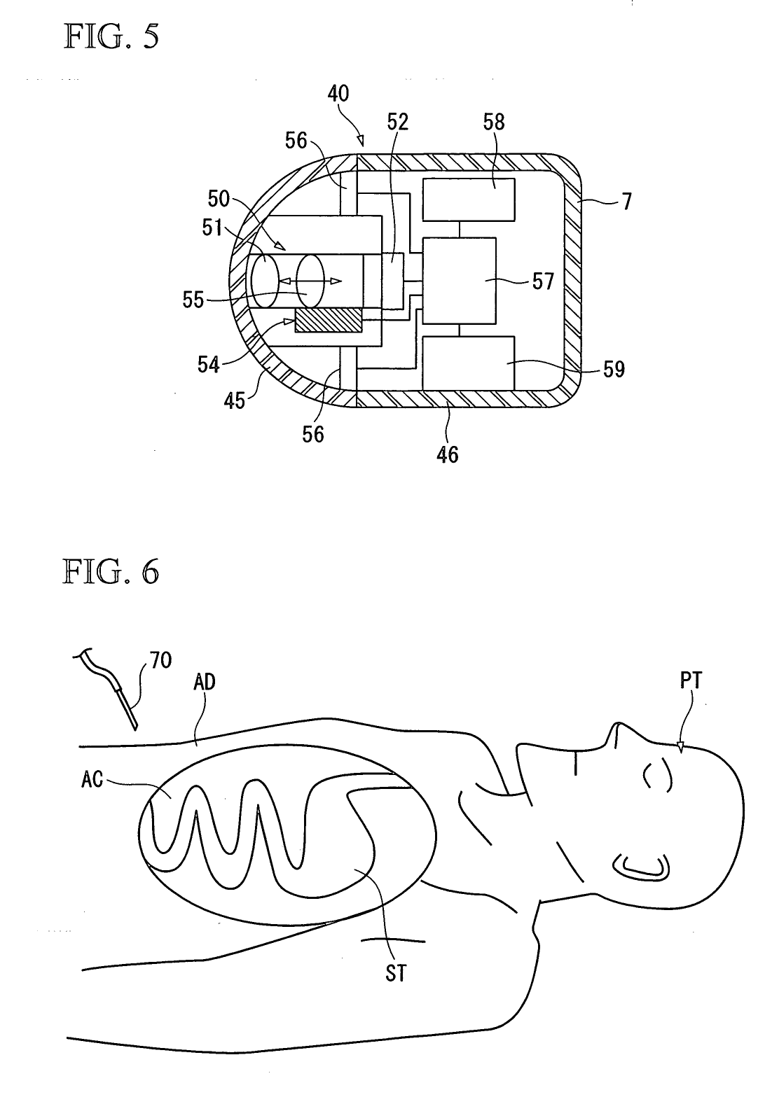

As shown in FIG. 5, in the casing 46, a second observation portion 50 serving as an observational optical device is arranged toward the transparent hood 45. The second observation portion 50 has an object lens 51 and an imager 52 arranged in the imaging position of the object lens 51, and is constructed so that the magnification can be changed by moving a zoom lens 55 by means of a zoom mechanism 54. The second observation portion 50 preferably comprises the zoom mechanism 54, however the zoom mechanism 54 is not an essential component in the present embodiment. Around the second observation portion 50 is arranged an illumination part 56 so as to illuminate the view field of the second observation portion 50. For the illumination part 56, for example a plurality of LEDs (Light Emitting Diodes) are used. The second observation portion 50 and the illumination part 56 are connected to a control circuit 57. The control circuit 57 is provided with a camera control unit (CCU) connected to the imager 52, a circuit which turns on the illumination part 56, and so forth. Furthermore, the control circuit 57 is connected with an antenna 58 and a battery 59.

The capsule endoscope 40 obtains an observation image (endoscopic image) under light of the illumination part 56. The observation image is converted into electric signals and output to the control circuit 57 by the imager 52. The control circuit 57 sends the electric signals of the observation image to the antenna 58, which oscillates them toward outside of the body as radio signals. The receiver 60 shown in FIG. 1 receives these radio signals and outputs to the controller 24 of the endoscope. That is, an image of the capsule endoscope 40 (hereunder, called a second observation image) can be displayed on a monitor 25 via the receiver 60. Detailed description of the capsule endoscope is disclosed in International Patent Application WO02004/112593. The contents disclosed in the International Patent Application WO2004/112593 are incorporated in the present embodiment.

The operation of the present embodiment is described. Hereunder is a description of a procedure for treating an organ or a tissue (hereunder, called a target site) serving as an object on which a desired medical procedure is performed, by inserting the endoscope 1 from a patient's mouth as a natural orifice of a living body. However, the natural orifice to be inserted with the endoscope 1 is not limited to the mouth, and may be a nostril or the anus. Moreover, treatments as a medical procedure are applicable to various actions such as suture, observation, incision, and cell sampling.

As shown in FIG. 6, a patient PT is laid on his back so that the abdomen AD is uppermost. Then a pneumoperitoneal needle 70 is pierced into the abdomen AD, and carbon dioxide gas or the like is sent into the abdominal cavity AC to expand the abdominal cavity. It is desirable to expand the abdominal cavity in order to ensure a space for performing a medical procedure in the abdominal cavity, however a pneumoperitoneum is not necessarily performed as long as a desired space can be ensured. Moreover, a method of expanding the abdominal cavity is not limited to a method of expanding by a gas, and may be a publicly known lifting method so as to keep a space in the abdominal cavity. Moreover, the timing to expand the abdominal cavity may be after a device (for example, the endoscope 1) is introduced into the abdominal cavity.

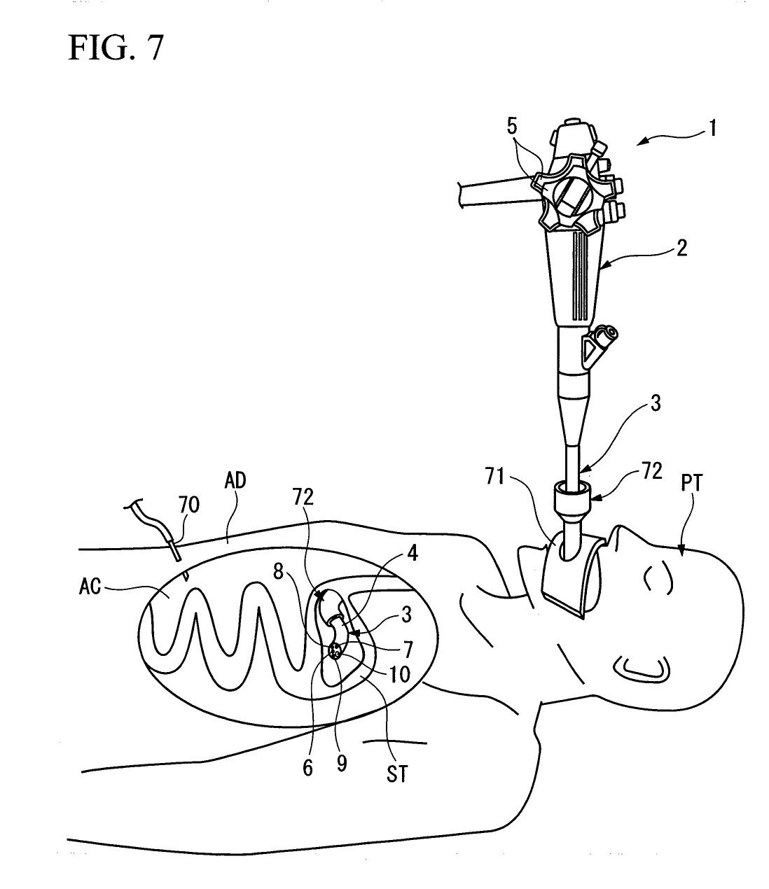

As shown in FIG. 7, after the abdomen AD of the patient PT is expanded by means of pneumoperitoneum, the endoscope 1 is passed through from a mouthpiece 71 attached to the patient PT's mouth into the body. Preferably, as shown in FIG. 7, upon insertion of the endoscope 1 into the body, an overtube 72 is used in common. The overtube 72 is used as a guide tube for inserting/withdrawing a device having an insertion portion such as the endoscope 1, into/from the body. However, the device may be inserted into the body without using this.

The endoscope 1 at this time is not attached with the capsule endoscope 40. After the insertion portion 3 is inserted into the stomach ST, the fluid supply channel 8 is used to send a gas into the stomach ST so as to expand the stomach ST. A device for incision, for example a high frequency knife, is passed through the work channel 10, and the stomach wall is incised. The incision site is desirably an anterior wall of the stomach.

After the stomach wall is incised, the endoscope 1 is withdrawn from the body, and then the grasping forceps 30 are passed through the work channel 10. After the treatment portion 35 is projected from the distal face 3A of the insertion portion 3, the handle 32 of the forceps operation portion 31 is moved back and forth to open/close the pair of forceps members 37, so as to hold the capsule endoscope 40. As shown in FIG. 3, if the grasping forceps 30 is moved backward, the magnetic bodies 47 on the proximal end of the capsule endoscope 40 are abutted against the distal face 3A of the insertion portion 3.

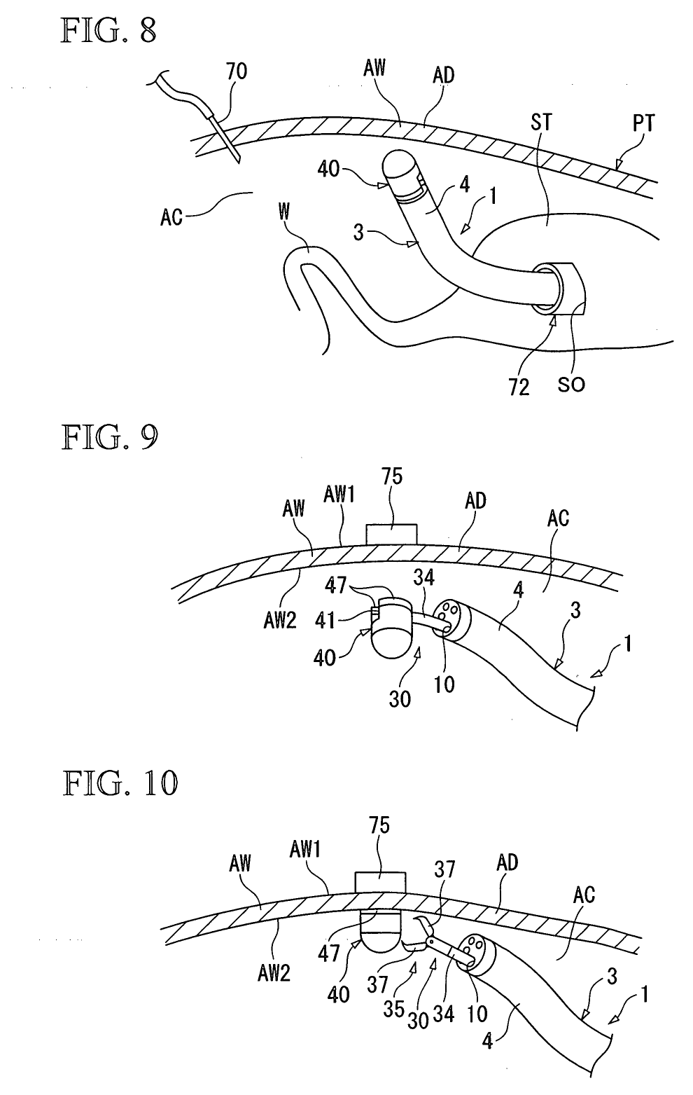

While the capsule endoscope 40 is held, the endoscope 1 is inserted from the patient PT's mouth into the stomach ST. For the image of inside the body, an image captured by the second observation portion 50 of the capsule endoscope 40 is used. Moreover, as shown in FIG. 8, the insertion portion 3 is introduced from an orifice SO formed by incision in the stomach wall, into the abdominal cavity AC. At this time, preferably, the target site W is confirmed by the capsule endoscope 40.

Next, the insertion portion 3 is curved toward the abdominal wall AW. In the vicinity of the abdominal wall AW, the grasping forceps 30 is moved forward with respect to the endoscope 1, and the capsule endoscope 40 is separated from the endoscope 1. As shown in FIG. 9, the capsule endoscope 40 is rotated about the pin 42 (refer to FIG. 3) with respect to the grasping forceps 30, and hung from the grasping forceps 30. As a result, the magnetic bodies 47 go toward the abdominal wall AW. In the present embodiment, the magnetic bodies 47 are provided on the proximal end of the capsule endoscope 40 (the opposite side to the view field direction of the second observation portion 50). Moreover, the construction is such that the magnetic bodies 47 go toward the abdominal wall AW, however it is not limited to this. For example, the construction may be such that a magnetic body is arranged in an optional position of the capsule endoscope 40, such as a side of the capsule endoscope 40, and a magnetic force is generated between this magnetic body and a magnetic body set outside of the body, so as to obtain an image of the abdominal cavity at a desired angle.

A magnet 75 is put on an outer surface AW1 (also called the abdomen or the abdomen outer surface) of the abdominal wall AW. While confirming that the target site W can be observed by the capsule endoscope 40 on the display of the monitor 25, the magnetic bodies 47 of the capsule endoscope 40 are attracted to the magnet 75 having the abdominal wall AW therebetween. The magnet 75 is used for placing the capsule endoscope 40 on the abdominal wall AW using the magnetic force effect. In the present embodiment, a permanent magnet is used as the magnet 75, however an electromagnet may be used.

As a result, as shown in FIG. 10, the capsule endoscope 40 is attached to the inner surface AW2 of the abdominal wall AW, and does not drop even if the grasping forceps 30 is opened. The magnet 75 may be previously put in the vicinity of the target site W of the abdominal wall AW, or may be disposed while the position of the capsule endoscope 40 is being searched.

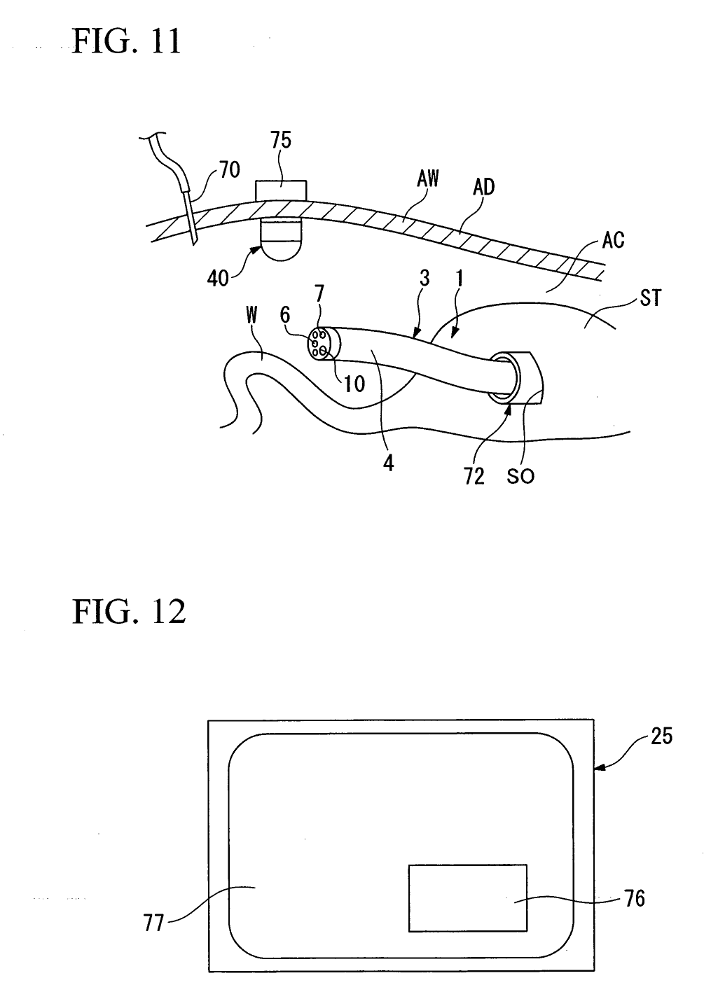

After the grasping forceps 30 is detached from the capsule endoscope 40, the insertion portion 3 is curved again. As shown in FIG. 11, while searching for the target site W by images of the first observation device 6 provided on the insertion portion 3, and the capsule endoscope as the second observation device, the endoscope 1 is moved forward to the target site W. The image of the first observation device 6 is a localized image, whereas the image of the capsule endoscope 40 is an image of a wide area where the distal end 4 of the insertion portion 3 enters, that is, an overhead view of the target site W. Therefore, by watching these two images, the position of the insertion portion 3 and the position of the target site W can be ascertained.

Here, as shown as an example in FIG. 12, if image processing is performed to display an image 76 of the first observation device 6 and an image 77 of the capsule endoscope 40 superposed in the monitor 25, all that an operator has to do is simply to confirm the image on one monitor 25. In this monitor 25, the display is such that the image 76 of the capsule endoscope 40 is superposed on a part of the image 77 of the first observation device 6 of the endoscope 1, and the overall image can be readily ascertained by confirming these two images. These images 76 and 77 can be switched by operating the buttons 23 of the endoscope 1. These two images 76 and 77 may be displayed in two split screens, instead of being displayed by partial superposition. Moreover, the images 76 and 77 may be separately displayed in two monitors. Furthermore, while a medical procedure is performed in the abdominal cavity AC, the magnet 75 outside of the body may be operated to move the position and the view field direction of the capsule endoscope 40 as the second observation device, so as to obtain an informative image as the second observation image.

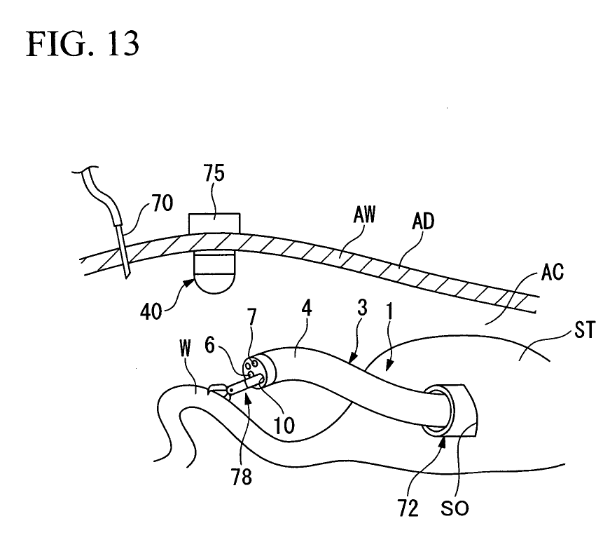

After the insertion portion 3 is faced to the target site W, the grasping forceps 30 is withdrawn, and a treatment tool is passed through the work channel 10 instead. For example, if a resection forceps 78 as shown in FIG. 13 is used, then while watching-the image of the first observation device 6 and the image of the capsule endoscope 40 as the second observation device, a tissue of the target site W is resected. If the image 76 of the first observation device 6 is largely displayed, since an enlarged image of the target site W and the resection forceps 78 can be obtained, the operation is facilitated. The construction may be such that the size of the image 77 of the first observation device 6 and the size of the second image 76 (second observation image) of the second observation portion 50 of the capsule endoscope 40 can be selectively switched according to the operation of the operator. For example, the construction may be such that when the device is made to approach the target site, the image 77 of the first observation device is displayed on a part of the second image 76 of the second observation device, and then when a medical procedure is performed, the image processing is switched so that the second image 77 of the second observation portion 50 is displayed on a part of the image 77 of the first observation device 6.

If the treatment of the target site W is incision of a tissue for example, then after the tissue is incised, a treatment tool for suture is passed through the endoscope 1 and the incised orifice is sutured. Then, the grasping forceps 30 is passed through the endoscope 1 again, and the capsule endoscope 40 is collected.

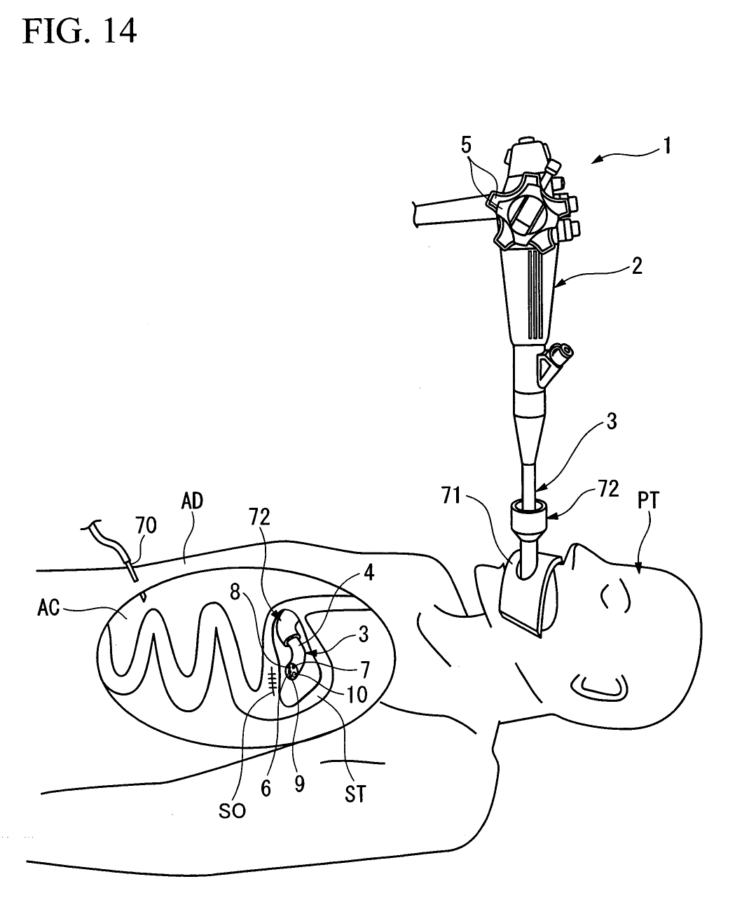

Specifically, the treatment portion 35 of the grasping forceps 30 is inserted into the groove 41 in the capsule endoscope 40, and the pair of forceps members 37 are opened/closed so as to hold the pin 42. After the holding of the capsule endoscope 40 is confirmed by the first observation device 6, the magnet 75 outside of the body is removed. Since the capsule endoscope 40 comes off from the inner surface AW2 of the abdominal wall AW and is hung from the grasping forceps 30, if the grasping forceps 30 is moved backward, the magnetic bodies 47 of the capsule endoscope 40 are abutted against the distal face 3A of the insertion portion 3. In this state, the endoscope 1 is brought back into the stomach ST from the orifice SO in the stomach wall and withdrawn from the patient PT's mouth, and then the capsule endoscope 40 is taken out to outside of the body. Furthermore, the endoscope 1 having the treatment tool for suture passed therethrough is inserted from the mouth again, and the orifice SO in the stomach wall is sutured. As shown in FIG. 14, after the suture of the orifice SO is completed, the endoscope 1 is withdrawn from the patient, and the pressure on the abdominal cavity AC is removed, after which the pneumoperitoneal needle 70 is withdrawn, and the procedure is completed. The timing when the capsule endoscope 40 serving as the second observation device is taken out from the body, is set before suture, however it may be brought back into the stomach ST before the suture, and taken out from the stomach ST after the suture.

According to the present embodiment, when a procedure is performed by introducing the endoscope I from the mouth through the stomach ST into the abdominal cavity AC, the capsule endoscope 40 that can be used apart from the endoscope 1 is arranged as the second observation device on the inner surface AW2 of the abdominal wall AW. Therefore, an image in a wide view field can be obtained. Since images including the target site W and the insertion portion 3 from different angles can be obtained, the operator can readily ascertain the positional relation, the direction, and the movement of respective sites, thus facilitating the procedure. Particularly when it is difficult to identify the target site W in. the view field of the first observation device 6, in a conventional method and device construction, the endoscope 1 has ,been operated to move the first observation device 6 to change the angle and the view field so as to identify the target site W, thus loading a burden onto the operator and the patient. However, the present embodiment can solve such a problem.

When the abdomen AD of the patient PT is faced upwards, the capsule endoscope 40 is arranged on the inner surface AW2 of the abdominal wall AW. Therefore, the image of the capsule endoscope 40 becomes an image as if the operator overlooks the insertion portion 3, the treatment tool, and the target site W. Furthermore, the procedure is further facilitated by adjusting the direction of the capsule endoscope 40 or performing image processing so that the vertical, sideways, and lengthwise directions recognized by the operator are matched with the actual directions.

Since the capsule endoscope 40 is arranged. on the inner surface AW2 of the abdominal wall AW by using the magnetic bodies 47, a burden is not loaded on the patient. Since the capsule endoscope 40 can be arranged and detached by simply putting on/taking off the magnet 75 outside of the body, the operation is facilitated. In particular, complicated operations become unnecessary on the inner surface AW2 side of the abdominal cavity AD.

The magnet 75 may be an electromagnet. The capsule endoscope 40 may have a sucker instead of the magnetic bodies 47. By attaching it onto the inner surface AW2 of the abdominal wall AW by means of a sucker, a similar effect to the above can be obtained. Moreover, a recess may be provided in the outer periphery of the proximal end of the capsule endoscope 40 so as to attach a clip to clamp the inner surface AW2 of the abdominal wall AW to the recess. The clip enables the capsule endoscope 40 to be fixed to the abdominal wall AW, and a similar effect to the above can be obtained. The clip is passed through the work channel as a treatment tool. Moreover, the clip may be previously and integrally attached onto the outer periphery of the capsule endoscope 40.

### SECOND EMBODIMENT

A second embodiment is described in detail with reference to the drawings.

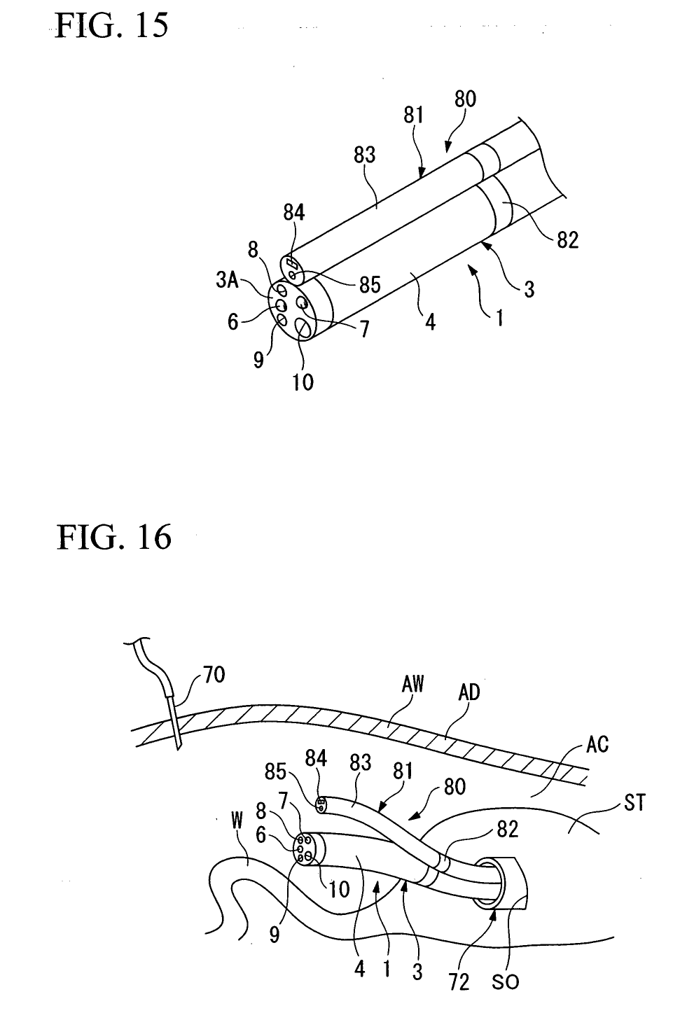

As shown in FIG. 15, the insertion portion 3 of the endoscope 1 is fixed with a small scope 80 as a second observation device in the present embodiment, by a connection member 82. The small scope 80 is an endoscope exclusively for observation, having an illuminating device 84 and a second observation device 85 on the-distal end of a flexible insertion portion 81, but not having various channels so as to decrease the diameter. The insertion portion 81 is extended along the insertion portion 3 and is connected by the connection member 82. The distal side from the part connected by the connection member 82 becomes a curvable portion 83. That is, the position where the insertion portion 81 is fixed to the insertion portion 3 is the proximal side from the curvable portion 83, being a position not interfering with curving operations of the respective curvable portions 4 and 83. The curving operation is performed by an operation portion on the hand side. The diameter of the insertion portion 81 is sufficiently narrow compared to the diameter of the insertion portion 3, being a size not interfering with insertion/withdrawal of the endoscope 1. The illuminating device 84 and the second observation device 85 of the small scope 80 have, for example similar constructions to those of the illuminating device 7 and the first observation device 6 of the endoscope 1, and are connected to the controller 24 through the inside of the small scope 80.

The operation of the present embodiment is described.

The endoscope 1 attached with the small scope 80 is inserted from the patient PT's mouth into the stomach ST. A high frequency knife is passed through the work channel 10 of the endoscope 1, and the stomach wall is incised to form the orifice SO. The endoscope 1 is introduced from the orifice SO into the abdominal cavity AC, and the target site W is confirmed by using the first observation device 6 and the second observation device 85. At this time, as shown in FIG. 16, if the second observation device 85 is moved in a direction away from the first observation device 6 by curving the curvable portion 83 of the small scope 80, two images from different viewpoints are obtained.

While confirming these two images, the operator makes the insertion portion 3 face toward the target site W. The forceps is passed through the work channel 10 of the endoscope 1, and the target site W is treated while confirming the two images. The two images obtained by the first observation device 6 and the second observation device 85 may be displayed either simultaneously or selectively. After the treatment in the abdominal cavity AC is completed, the curvable portion 83 of the small scope 80 is brought back along the insertion portion 3, and then the insertion portion 3 is withdrawn from the abdominal cavity AC back into the stomach. The treatment tool for suture is passed through the work channel 10, and the orifice SO in the stomach wall is sutured, and then the endoscope 1 is withdrawn from the body.

According to the present embodiment, when a procedure is performed by introducing the endoscope 1 from the mouth through the stomach ST into the abdominal cavity AC, the small scope 80 along the insertion portion 3 is used, enabling to arrange the second observation device 85 away from the first observation device 6 at a predetermined distance. Since two images from different angles can be obtained, the operator can readily ascertain the positional relation, the direction, and the movement of respective sites, further facilitating the procedure. If the construction is such that the second observation device 85 is arranged in a position back from the first observation device 6, or that a wide-angle lens is attached to the second observation device 85, so as to enable to observe a wider view field than that of the first observation device 6, then an image from a wide view field can be obtained as a second observation image, flurther facilitating the procedure in this case too.

Similarly to the first embodiment, when a medical procedure is performed in the abdominal cavity AC, a curving operation of the curvable portion 83 of the small scope 80 and/or back-and-forth moving operation of the small scope 80 may be performed so as to move the position of second observation device 85 to a desired location (to change the viewpoint of the second observation device 85).

Since the second observation device 85 is not completely separate from the endoscope 1, the recovery operation can be omitted. Moreover, the position of the second observation device 85 can be readily changed in the middle of procedure, enabling to obtain optimum images according to the type of procedure and its progress.

### THIRD EMBODIMENT

A third embodiment is described in detail with reference to the drawings. The present embodiment is characterized in using an overtube (also called a guide tube or a device) for passing an endoscope therethrough.

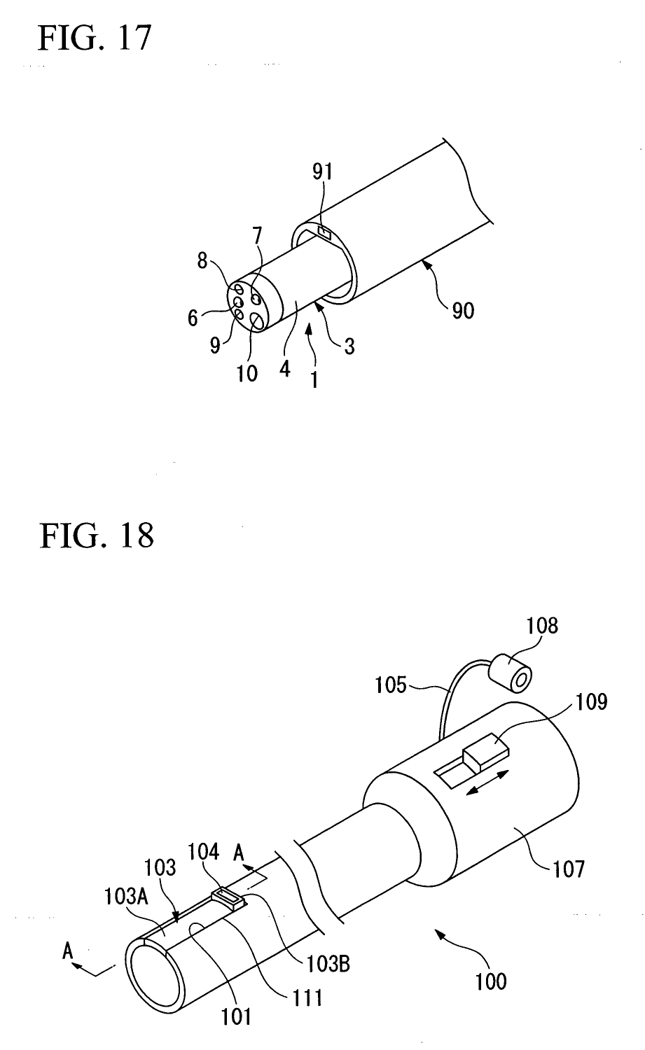

As shown in FIG. 17, an overtube 90 is made from a flexible and slender barrel, inside of which the endoscope 1 can be inserted in a back-and-forth movable manner. The distal end of the overtube 90 is attached with a second observation device 91 having an image-capturing face facing forward. This second observation device 91 captures an image in a range illuminated by the illuminating device 7 on the endoscope 1 side, however another illuminating device may be arranged around the second observation device 91.

When a procedure is performed, the endoscope 1 is guided into the abdominal cavity AC together with the overtube 90, and the target site W is confirmed by respective images of the first observation device 6 and the second observation device 91. After the distal end of the endoscope 1 is pushed out from the overtube 90, the procedure is performed. The first observation device 6 provided on the endoscope 1 obtains an image from a position close to the treatment tool and the target site W. Since the second observation device 91 provided on the overtube 90 is in a position away from the first observation device 6, it obtains an image of a wide view field including the distal end of the endoscope 1, the treatment tool, and the target site W. The operator performs the procedure while simultaneously or selectively confirming two images in different image-capturing positions.

In the present embodiment, since the procedure can be performed using two images captured from different points in the insertion direction of the endoscope 1, the positional relation between the target site W and the treatment tool are readily confirmed, facilitating the procedure. Since the second observation device 91 is provided on the distal end of the overtube, positioning is easy. The size of the view field of the second observation device 91 can be readily adjusted by relatively changing the distance from the distal area of the overtube 90 to the target site W. Such an adjustment of distance can be achieved by the projected amount of the endoscope 1 from the overtube 90. In the present embodiment, similarly to the first and second embodiments, when a desired medical procedure is performed in the abdominal cavity AC, rotation and/or back-and-forth moving operation of the overtube 90 may be performed so as to change the position of the second observation device 91 to a desired condition (to change the viewpoint of the second observation device 91).

### FOURTH EMBODIMENT

A fourth embodiment is described in detail with reference to the drawings. The present embodiment is characterized in that the distal end of an overtube is attached with a second observation device in a positionally adjustable manner.

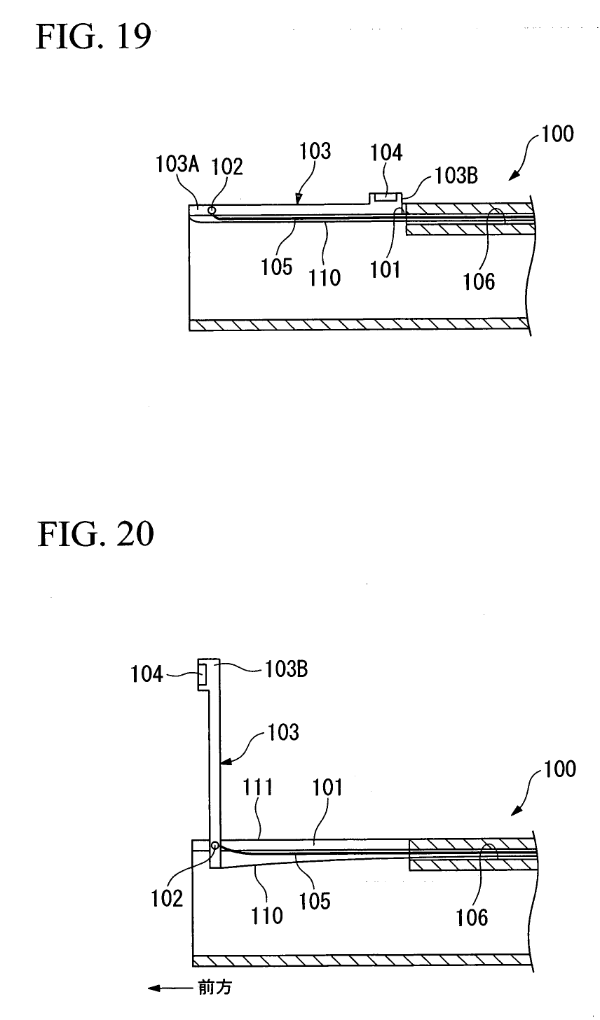

As shown in FIG. 18 and FIG. 19, on the distal end of an overtube 100, a slit 101 is formed slenderly along the lengthwise direction. The distal end side of the slit 101 is fixed with a pin 102 so as to transverse the slit 101. This pin 102 is attached with a proximal end 103A of a rising member 103. The rising member 103 has a shape to fit in the slit 101, and in a condition where it is accommodated in the slit 101, the outline of the overtube 100 is hardly changed. The distal end 103B of the rising member 103 is provided with a second observation device 104 oriented radially outward. An electric signal output from the second observation device 104 is output to a cable 105. The cable, 105 is led out from the vicinity of the pin 102, and led out through a lumen 106 on the inner peripheral side of the overtube 100, to a proximal end 107 on the hand side. The cable 105 is led out from the proximal end 107, and the construction is such that an image can be obtained by connecting a connector 108 to the controller 24 (refer to FIG. 1). Furthermore, the proximal end 107 is provided with a slide member 109. The slide member 109 is slidable in the lengthwise direction of the overtube 100, and a wire 110 is fixed inside thereof. The wire 110 is guided through the lumen 106 of the overtube 100 to the distal end, and fixed to a distal side further from the pin 102 on the proximal end 103A of the rising member 103. The wire 110 has a flexibility but a predetermined rigidity, and is capable of raising the rising member 103 and pulling it into the slit 101, by moving the wire 110 back-and-forth.

When a procedure is performed, the overtube 100 and the endoscope 1 are guided through the orifice SO formed in the stomach ST, into the abdominal cavity AC. After the overtube 100 and the endoscope 1 are guided into the abdominal cavity AC so that the distal end of the overtube 100 reaches a desired position, the operator moves the slide member 109 backward. The wire 110 pulls the proximal end 103A of the rising member 103, to rotate the rising member 103 about the pin 102. As shown in FIG. 20, the rising member 103 rises so as to separate the second observation device 104 from the side face 111 of the overtube 110, and stops in a position approximately orthogonal to the lengthwise direction of the overtube 100. The second observation device 104 is arranged in a position away from the side face 111 of the overtube 100, and its observation view field direction is oriented forward in the insertion direction of the overtube 100 into the body. In this manner, by changing the position of the second observation device 104, an image obtained by the second observation device 104 becomes similar to an image in a condition where the target site W and the treatment tool are overlooked from the rear. The operator performs the procedure while confirming two images having different image-capturing positions.

In the present embodiment, since the procedure can be performed using two images captured from different points in the insertion direction of the endoscope 1, the positional relation between the target site W and the treatment tool are readily confirmed, facilitating the procedure. Since the second observation device 104 can be arranged in the position away from the overtube 100, an image can be obtained from a different angle and a different distance from those of an image of the first observation device 6, facilitating the confirmation of position of the treatment tool and the like. The image of the second observation device 104 becomes an image as if the target site W of the patient PT lying on his back is overlooked. Therefore the operator can readily and sensuously specify the positional relation. The range of the second observation image obtained by the second observation device 104 can be readily adjusted by relatively moving the position of the distal area of the overtube 100 with respect to the endoscope 1 and the target site W. The second observation device 104 can be pulled in to fit the outline of the overtube 100, when it is not used. Therefore the insertion/withdrawal can be smoothly performed without enlarging the outer diameter of the overtube 100.

In the present embodiment, similarly to the abovementioned embodiments, when a desired medical procedure is performed in the abdominal cavity AC, rotation and/or back-and-forth moving operation of the overtube 100 may be performed so as to change the position of the second observation device 104 to a desired condition (to change the viewpoint of the second observation device 104).

As mentioned above, while preferred embodiments of the present invention have been described, the present invention is not limited to these. Additions, omissions, substations of the construction, and other modifications can be made without departing from the scope of the present invention. The present invention is not to be considered as being limited by the forgoing description, and is only limited by the scope of the appended claims.

For example, the arrangement may be such that only an image of the second observation device is used when the insertion portion 3 is faced to the target site W, and only an image of the first observation device 6 is used when an actual procedure is performed.

If the endoscope 1 has a plurality of work channels 10, procedure can be performed using a plurality of treatment tools at the same time, improving the treatment properties.

When the endoscope 1 is inserted into the abdominal cavity AC, the arrangement may be such that the endoscope 1 is inserted from a natural orifice of the living body into another hollow organ, not limiting to the stomach ST, and then an orifice is formed in the wall of the hollow organ, so as to introduce the endoscope into the abdominal cavity AC.

In the first embodiment, when the capsule endoscope 40 is introduced into the abdominal cavity AC, the arrangement may be such that an exclusive introducing device is used, and after the capsule endoscope 40 is arranged, the exclusive introducing device is withdrawn from the body, and the endoscope 1 is inserted instead. In the introducing device in this case it is sufficient to have a construction without the first observation device 6. Moreover, the introducing device may have a construction where a holding part is fixed to the distal end, without having the work channel 10 capable of exchanging treatment tools.

The procedure may be performed by selectively displaying only one out of two images. For example, only an image of the second observation device is used when the location is confirmed, and only an image of the first observation device is used during a procedure.

The hollow organ formed with the orifice SO is not limited to the stomach ST. For example, it may be an esophagus, a duodenum, a small intestine, a colon, a uterus, a bladder, and the like.

The device needed for performing a desired procedure is not limited to the endoscope comprising the observation device and the work channel described in the above embodiments. For example, there may be used a device (hereunder, called a treatment device for convenience) comprising a treatment portion for performing a desired treatment, on the distal side of the insertion portion to be inserted into the body, and provided with an operation portion capable of operating this treatment portion from outside of the body. If the treatment device is provided with an observation device, an image from the observation device may be used as a first observation image. Moreover, if the treatment device does not have an observation device, various modes may be considered such as using the abovementioned capsule endoscope in common. Furthermore, as another example of the abovementioned treatment device, there may be used a device comprising a lumen through which the treatment tool can be inserted, in the insertion portion, but not having an observation mechanism.

What is claimed is:

1. A medical procedure through a natural orifice, comprising:
forming an orifice in a hollow organ by using a device inserted into the hollow organ from a natural orifice of a patient;
introducing a first observation device from said orifice formed in said hollow organ into an abdominal cavity;
introducing a second observation device from said orifice formed in said hollow organ into the abdominal cavity;
arranging said second observation device in a position different from that of said first observation device;
simultaneously or selectively displaying an image obtained from said first observation device and an image obtained from said second observation device; and
performing a desired procedure in the abdominal cavity while confirming the images.

2. The medical procedure through a natural orifice according to claim 1, wherein displaying an image includes sending an image signal of an image in an abdominal cavity obtained by the second observation device, to the outside of the body by a wireless communication.

3. The medical procedure through a natural orifice according to claim 1, wherein arranging said second observation device in a position different from that of said first observation device includes arranging a view field direction of said second observation device that has been set on an inner surface of an abdominal wall of a patient laid on his back, toward an organ in the abdominal cavity.

4. The medical procedure through a natural orifice according to claim 3, wherein arranging said second observation device in a position different from that of said first observation device includes arranging a device generating a magnetic force on the outside an abdominal wall so as to fix said second observation device to the inside of the abdominal wall, by a magnetic force.

5. The medical procedure through a natural orifice according to claim 1, comprising sending a gas into an abdominal cavity of a patient to expand the cavity.

6. The medical procedure through a natural orifice according to claim 1,
wherein forming an orifice in a hollow organ includes forming said orifice by observing an image obtained by said first observation device provided on said device having an insertion portion to be inserted into the body, and
wherein introducing said first observation device into an abdominal cavity includes introducing a device comprising said first observation device through said orifice into the abdominal cavity.

7. The medical procedure through a natural orifice according to claim 1, wherein introducing said second observation device into an abdominal cavity includes using said device to introduce an observation device that can be placed in the body and constituted to be capable of sending an image signal of an obtained image to the outside of the body by means of a wireless communication, into the abdominal cavity.

8. The medical procedure through a natural orifice according to claim 6, wherein forming an orifice in a hollow organ by using said device includes inserting a treatment tool into a work channel of said device having said work channel provided on an insertion portion to be inserted into the body, and forming said orifice by this treatment tool.

9. The medical procedure through a natural orifice according to claim 6, wherein introducing said second observation device into an abdominal cavity includes introducing a second device comprising said second observation device provided on an insertion portion to be inserted into the body, through said orifice into the abdominal cavity.

10. The medical procedure through a natural orifice according to claim 9, wherein arranging said second observation device in a position different from that of said first observation device includes curving a distal area of said second device, so as to move said second observation device to a desired position away from said first observation device.

11. The medical procedure through a natural orifice according to claim 10, wherein arranging said second observation device in a position different from that of said first observation device includes curving a distal area of said second device in order to change a viewpoint of said second observation device, so as to move said second observation device to a desired position, when a desired procedure is performed in an abdominal cavity.

12. The medical procedure through a natural orifice according to claim 6, wherein introducing said second observation device into an abdominal cavity includes introducing a distal end of a guide tube which comprises said second observation device at the distal end thereof and is capable of inserting said device to the inside, into the abdominal cavity.

13. The medical procedure through a natural orifice according to claim 12, wherein arranging said second observation device in a position different from that of said first observation device includes observing an image obtained from said second observation device provided on said guide tube, so as to adjust a relative position of the distal area of said guide tube with respect to said device.

14. The medical procedure through a natural orifice according to claim 13, wherein arranging said second observation device in a position different from that of said first observation device includes operating said guide tube from the outside of the body in order to change a viewpoint of said second observation device, so as to change a position of said second observation device, when a desired procedure is performed in an abdominal cavity.

15. The medical procedure through a natural orifice according to claim 12, wherein arranging said second observation device in a position different from that of said first observation device includes operating said guide tube from the outside of the body to raise a rising member provided with said second observation device, so as to move said second observation device in a direction away from the side face of said guide tube.
